(19) United States
(12) Patent Application Publication
(10) Pub. No.: US 2022/0169626 A1
(43) Pub. Date: Jun. 2, 2022

(54) CANNABICHROMENE-O-ACETATE SYNTHESIS, COMPOSITIONS, AND METHODS OF USE

(71) Applicant: Marquette Analytica, Midland, MI (US)

(72) Inventor: Robert Opperman, Midland, MI (US)

(73) Assignee: Marquette Analytica, Midland, MI (US)

(21) Appl. No.: 17/456,695

(22) Filed: Nov. 29, 2021

Related U.S. Application Data
(60) Provisional application No. 63/119,967, filed on Dec. 1, 2020.

(51) Int. Cl. C07D 311/58

(52) U.S. Cl. CPC C07D 311/58; A61K 47/44

(57) ABSTRACT

A cannabichromene derivative, cannabichromene-o-acetate, synthesis, compositions comprising the same, and methods of use.

### CROSS-REFERENCE TO RELATED APPLICATIONS

This patent application claims priority to U.S. Provisional Patent Application No. 63/119,967, filed on December 1, 2020, the disclosure of which is herein incorporated in its entirety.

### FIELD OF THE INVENTION

This disclosure relates to cannabichromene-o-acetate (CBC-o-acetate), compositions, and methods of use thereof.

### BACKGROUND OF THE INVENTION

Cannabis has been used as a source of fiber to make paper and clothing, as a recreational drug, and in traditional medicine. In recent years, compounds present in Cannabis, including the cannabinoids Δ⁹-tetrahydrocannabinol (THC) and cannabidiol (CBD), have been shown to alleviate inflammation and cancer-related symptoms. See, e.g., Federica Pellati et al., Cannabis sativa L. and Nonpsychoactive Cannabinoids: Their Chemistry and Role against Oxidative Stress, Inflammation, and Cancer, BIOMED RES. INT'L, 2018.

Historically, Cannabis has been divided into three species: Cannabis sativa, Cannabis indica, and Cannabis ruderalis. These species have been extensively hybridized, and the resulting hybrids are classified as C. sativa and can be further characterized by chemotype according to the cannabinoid profile. Cannabis plants having high THC levels are often used for medicinal properties. In contrast, Cannabis plants having low THC levels (hemp) and high CBD levels have been used in textiles and foods. Cannabichromene (CBC) is a cannabinoid present in cannabis. There exists a need for efficient cannabinoid compositions for use in medicine.

### SUMMARY

The disclosure provides, among other things, methods of preparing cannabichromene derivatives, compositions, and methods of use.

In an embodiment, cannabichromene-o-acetate (CBCOA).

In an embodiment, a composition may comprise cannabichromene-o-acetate (CBCOA). The composition may comprise between about 1 ng and 1,000 ng of cannabichromene-o-acetate (CBCOA). The composition may comprise between about 1 mg and 1,000 mg of cannabichromene-o-acetate (CBCOA). The composition may comprise between about 1 g and 1,000 g of cannabichromene-o-acetate (CBCOA).

In an embodiment, a compound of Formula I:

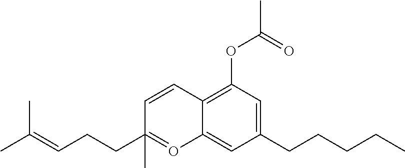

In an embodiment, a composition may comprise the compound of Formula I. The composition may comprise between about 1 ng and 1,000 ng of the compound of Formula I. The composition may comprise between about 1 mg and 1,000 mg of the compound of Formula I. The composition may comprise between about 1 g and 1,000 g of the compound of Formula I.

In an embodiment, a compound of Formula II:

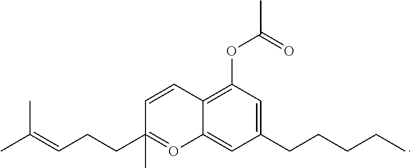

In an embodiment, a composition may comprise the compound of Formula II. The composition may comprise between about 1 ng and 1,000 ng of the compound of Formula II. The composition may comprise between about 1 mg and 1,000 mg of the compound of Formula II. The composition may comprise between about 1 g and 1,000 g of the compound of Formula II.

In an embodiment, a composition may comprise cannabichromene-o-acetate.

In an embodiment, a composition may further comprise an excipient, lubricant, antioxidant, emulsifier, thickening, stabilizer, solvent, diluent, buffer, vehicle, or a combination thereof

In an embodiment, a composition may further comprise a fatty acid, oil, or combination thereof. The fatty acid may be arachidic acid, arachidonic acid, behenic acid, brassidic acid, butyric acid, capric acid, caproic acid, caproleic acid, caprylic acid, cerotic acid, dihomo-γ-linolenic acid (DGLA), docosahexaenoic acid (DHA), docosapentaenoic acid (DPA), eicosapentaenoic acid (EPA), elaidic acid, eurcic acid, gadoleic acid, lauric acid, lauroleic acid, lignoceric acid, linoelaidic acid, linoleic acid, α-linoleic acid, γ-linoleic acid, mead acid, myristic acid, myristoleic acid, nervonic acid, oleic acid, palmitic acid, palmitoleic acid, pinolenic acid (columbinic acid), sapienic acid, stearic acid, vaccenic acid, α-linoleic acid, or a combination thereof.

In an embodiment, the oil may be a vegetable oil. In an embodiment, the vegetable oil may be soybean oil, corn oil, sunflower oil, hemp seed oil, coconut oil, olive oil, canola oil, cottonseed oil, palm oil, peanut oil, safflower oil, sesame oil, or a mixture thereof.

In an embodiment, the oil may be a nut oil. In an embodiment, the nut oil is almond oil, beech nut oil, cashew oil, hazelnut oil, macadamia oil, mongongo nut oil (manketti oil), pecan oil, pistachio oil, walnut oil, pumpkin seed oil, or a mixture thereof.

In an embodiment, the composition may comprise a mixture of at least two oils.

In an embodiment, the composition may further comprise a cannabinoid. In an embodiment, the cannabinoid may be cannabidiol, tetrahydrocannabinol (THC), cannabidiol (CBD), cannabidiol acid (CBDA), cannabigerol, cannabinol, cannabichromene, cannabigerivarin, tetrahydrocannabivarin, cannabidivarin, cannabichromevarin, or a mixture thereof.

In an embodiment, the composition may further comprise a cannabinoid acid selected from the group consisting of cannabigerolic acid, cannabidiol acid (CBDA), Δ9-tetrahydrocannabinolic acid (THCA), cannabidiolic acid, cannabichromenenic acid, cannabigerovarinic acid, tetrahydrocanabivarinic acid, cannabidivarinic acid, cannabichromevarinic acid, or a mixture thereof.

In an embodiment, the composition may further comprise a terpene.

In an embodiment, the terpene may be alpha bisabolol, alpha phellandrene, alpha pinene, beta caryophyllene, beta pinene, cadinene, camphene, camphor, citral, citronellol, delta 3 carene, eucalyptol, eugenol, gamma terpinene, geraniol, humulene, limonene, linalool, nerol, nerolidol, ocimene, para-cymene, phytol, pulegone, terpineol, terpinolene, valencene, or mixtures thereof.

In an embodiment, the composition may be substantially free of lipids. In an embodiment, the composition may comprise less than 1% lipids w/w. In an embodiment, the composition may comprise less than 0.3% tetrahydrocannabinol (THC) by weight (w/w). In an embodiment, the composition may comprise less than 0.1% tetrahydrocannabinol (THC) by weight (w/w). In an embodiment, the composition may be substantially free of terpenes. In an embodiment, the composition may comprise less than 0.1% to 1% terpenes by weight. In an embodiment, the composition may be substantially free of pesticides, fungicides, fertilizers, plant material, organelles, nucleic acids, lignin, and mixtures thereof. In an embodiment, the composition may comprise less than 1% w/w of pesticides, fungicides, fertilizers, plant material, organelles, nucleic acids, lignin, and mixtures thereof. In an embodiment, the composition may comprise less than 0.5%, 0.1%, 0.01%, or 0.001% w/w pesticides, fungicides, fertilizers, plant material, organelles, nucleic acids, lignin, and mixtures thereof. In an embodiment, the composition may comprise a concentration of pesticides or fungicides ranging from about 0 ppm to 10 ppm.

In an embodiment, the composition may be in the form of a tablet, pill, capsule, optionally an enteric capsule, liquid, syrup, gel, or gummy. In one embodiment, the composition may be in the form of a tincture. In an embodiment, the composition may be is a pharmaceutical composition. In an embodiment, the composition may be an edible composition.

In an embodiment, the composition may be a nutraceutical, foodstuff, or supplement.

In an embodiment, a method for treating cancer may comprise administering an effective amount of the compound of Formula I to a patient in need thereof. The cancer may be melanoma.

In an embodiment, a method for treating a patient with a tumor may comprise administering an effective amount of the compound of Formula I to a patient in need thereof.

In an embodiment, a method for treating cancer may comprise administering an effective amount of the composition comprising an effective amount of the compound of Formula I to a patient in need thereof. The cancer may be melanoma.

In an embodiment, a method for treating a patient with a tumor may comprise administering an effective amount of the composition comprising an effective amount of the compound of Formula I to a patient in need thereof.

In an embodiment, the composition may be administered intravesicularly, topically, orally, rectally, vaginally, topically, nasally, or via inhalation. In an embodiment, the composition may be administered orally, optionally in the form of a liquid. The composition may be formulated for oral administration.

In an embodiment, a method of preparing the compound of Formula I may comprise:
(a) heating cannabichromene to 95-105° C. to form a liquid,
(b) reacting at 95-105° C. the liquid from (a) with acetic anhydride to form a solution,
(c) optionally an acid to the solution from (b),
(d) cooling the solution from (b) or (c) to 20-25° C. to form a cooled solution,
(e) washing the cooled solution from (d) with water and hexane, forming organic and aqueous phases,
(f) separating the organic and aqueous phases, wherein the organic phase comprises the compound of Formula I; and
(g) recovering the compound of Formula I from the organic phase.

In one embodiment, a method of preparing the compound of Formula I may comprise:
(a) heating cannabichromene to 95-105° C. to form a liquid,
(b) reacting at 95-105° C. the liquid from (a) with acetic anhydride to form a solution,
(c) optionally an acid to the solution from (b),
(d) cooling the solution from (b) or (c) to 20-25° C. to form a cooled solution,
(e) washing the cooled solution from (d) with water and hexane, forming organic and aqueous phases,
(f) separating the organic and aqueous phases, wherein the organic phase comprises the compound of Formula II; and
(g) recovering the compound of Formula II from the organic phase.

In one embodiment, a method of preparing the compound of Formula I may comprise:
(a) heating cannabichromene to 95-105° C. to form a liquid,
(b) reacting at 95-105° C. the liquid from (a) with acetic anhydride to form a solution,
(c) optionally an acid to the solution from (b),
(d) cooling the solution from (b) or (c) to 20-25° C. to form a cooled solution,
(e) washing the cooled solution from (d) with water and hexane, forming organic and aqueous phases,
(f) separating the organic and aqueous phases, wherein the organic phase comprises cannabichromene-o-acetate (CBCOA); and
(g) recovering the cannabichromene-o-acetate (CBCOA) from the organic phase.

In an embodiment, the acid may be sulfuric acid, hydrochloric acid, nitric acid, hydrobromic acid, hydroiodic acid, perchloric acid, or chloric acid. In an embodiment, the acid may be sulfuric acid.

In an embodiment, one or more of steps (a), (b), (c), and (e) may further comprise stirring.

In an embodiment, in step (e), the water and hexane may be in a 1:1 ratio by volume. In an embodiment, in step (e), the water and hexane may be in a ratio of between about 1:1 to 1:100 by volume. In an embodiment, in step (e), the water and hexane may be in a ratio of between about 1:100 to 1:1 by volume.

In an embodiment, in step (b), the cannabichromene and acetic anhydride may be in a 1:1 molar ratio. In an embodiment, in step (b), the cannabichromene and acetic anhydride may be in a molar ratio of between about 1:1 to 1:100 by volume. In an embodiment, in step (b), the cannabichromene and acetic anhydride may be in a ratio of between about 1:100 to 1:1 by volume. In an embodiment, in step (b) the cannabichromene and acetic anhydride may be in a molar ratio of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, cannabichromene to acetic anhydride. In an embodiment, in step (b) the cannabichromene and acetic anhydride may be in a molar ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, acetic anhydride to cannabichromene. In other embodiments, another anhydride many be used.

In an embodiment, in step (f) cooling may comprise placing the organic phase from step (e) in a freezer capable of achieving −5° C., optionally lower.

In an embodiment, in step (g) comprises placing the organic phase comprising the compound of Formula I in a rotary evaporator.

In an embodiment, the cannabichromene is in the form of cannabichromene crystals.

In an embodiment, a use of the compound of Formula I for the manufacture of a medicament for the treatment of cancer, optionally melanoma.

In an embodiment, a use of the compound of Formula II for the manufacture of a medicament for the treatment of cancer, optionally melanoma.

In an embodiment, a composition for the treatment of cancer may comprise an effective amount of the compound of Formula I. In one embodiment, a composition for the treatment of a patient with a tumor may comprise an effective amount of the compound of Formula I.

In an embodiment, a composition for the treatment of cancer may comprise an effective amount of the compound of Formula II. In one embodiment, a composition for the treatment of a patient with a tumor may comprise an effective amount of the compound of Formula II.

In an embodiment, the composition may be formulated in the form of a tablet, pill, capsule, optionally an enteric capsule, liquid, syrup, gel, gummy, or tincture.

In an embodiment, the composition may be formulated for administration intravesicularly, topically, orally, rectally, vaginally, topically, nasally, or via inhalation.

### BRIEF DESCRIPTION OF THE DRAWINGS

FIG. 1A-B depict an exemplary structures of cannabichromene (CBC) (A) and cannabichromene-o-acetate (CBC-o-acetate) (B).

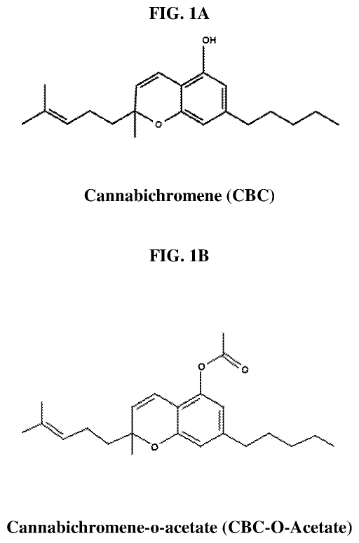

FIG. 2 depicts two exemplary reaction schemes for the production of cannabichromene-o-acetate from cannabichromene.

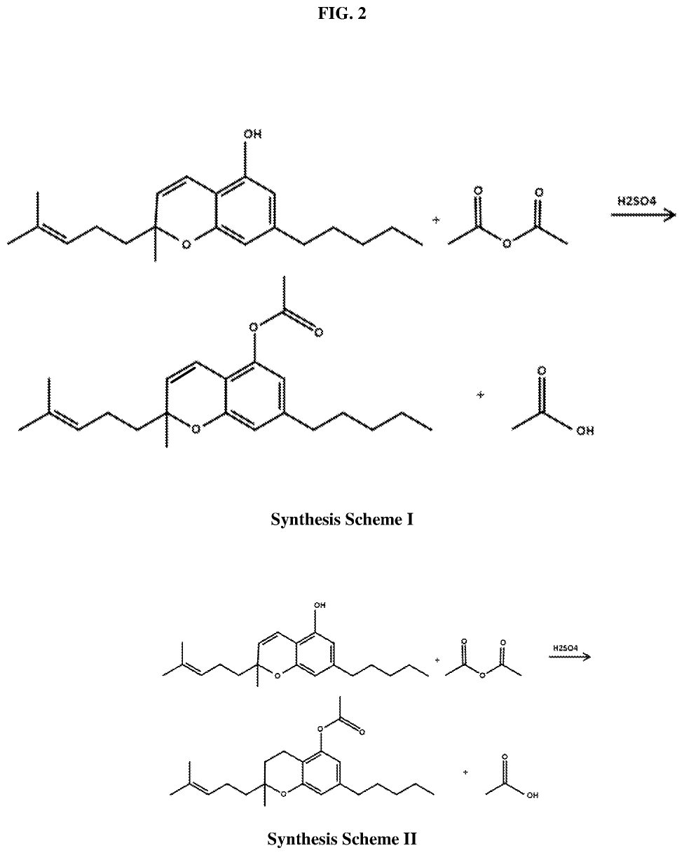

### Definitions

Unless defined otherwise, all technical and scientific terms used herein have the same meaning as those commonly understood by one of ordinary skill in the art.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the steps can be carried out in any order without departing from the principles of this disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

“Cannabis plant material,” “cannabis,” and “cannabis material,” as used herein, refers broadly to any cannabis plant or part thereof, this includes but is not limited to, flowers, stems, nodes, leaves, pistils, colas, calyxs, trichomes, seed, stalk, buds (including dormant buds, axillary buds, and terminal buds), petiole, rachis, bract, and roots. Cannabis plant material also refers broadly to hemp that includes but is not limited to cannabis plants with less than 0.3% THC content. Hemp and industrial hemp can be used interchangeably as both refer to cannabis plants with less than 0.3% THC content.

“Cannabis,” as used herein, refers broadly to all plants of the genus cannabis and/or the family cannabaceae, including but not limited to all plants of the species cannabis sativa, cannabis indica, and cannabis ruderalis. Hybrids, clones, cultivars, and varieties are also included. Cannabis also broadly includes hemp.

“Cannabis extract,” as used herein, refers broadly to any composition comprising a cannabinoid. Cannabis extracts may also comprise lipids, terpenes, solvent, or mixtures thereof.

“Cannabichromene (CBC),” as used herein, refers broadly to a cannabinoid with the following structure:

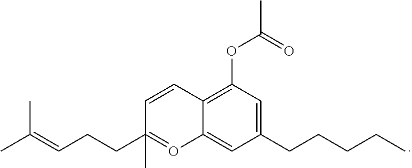

“Cannabichromene-o-acetate (CBC-o-acetate),” as used herein, refers broadly to a cannabichromene functionalized with an acetal group onto the hydroxyl group (Formula I):

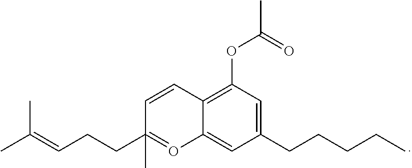

This compound may also be referred to as the compound of Formula II.

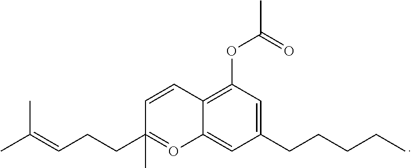

### DETAILED DESCRIPTION

### Medical Cannabis and Cannabinoids

Medical cannabis has been used to alleviate the symptoms of patients suffering from a variety of medical conditions including cancer, anorexia, AIDS, chronic pain, spasticity, glaucoma, arthritis, and migraines. For example, the antiemetic properties of Cannabis have been useful in the treatment of nausea and vomiting in cancer patients undergoing chemotherapy as well as in the treatment of weight loss syndrome associated with AIDS. Glaucoma patients have been treated with Cannabis to reduce intraocular pressure. Muscle relaxing and anticonvulsant effects of Cannabis have also been reported.

However, consumption of the whole Cannabis plant, e.g., by smoking, has also results in side-effect including impaired cognitive functions, perception, reaction time, learning, and memory. To mitigate such side-effects, there is growing interest in investigating the medicinal properties of individual Cannabis-derived compounds and sub-combinations and derivatives thereof. In addition, many potential patients have personal or religious objections to consuming cannabis in plant form. To address these issues, there is a great interest in developing a pharmaceutical form of cannabis extracts, especially crystallize extracts. The disclosure relates to methods of preparing a cannabinoid derivative that can be useful in such applications.

Cannabinoids are synthesized primarily in the glandular trichomes of Cannabis plants and include tetrahydrocannabinolic acid, Δ⁸ tetrahydrocannabinolic acid, Δ⁹ tetrahydrocannabinolic acid, tetrahydrocannabinol, Δ⁸ tetrahydrocannabinol, Δ⁹ tetrahydrocannabinol (THC), cannabidiolic acid, cannabidiol (CBD), cannabinol, cannabigerolic acid, cannabigerol, cannabigerolic acid, cannabichromene, and tetrahydrocannabivarin, any of which can be obtained by the methods known in the art and those methods and systems described in U.S. patent application Ser. No. 16/935,589, filed Jul. 22, 2020. Post-processing methods are described in U.S. patent application Ser. No. 17/025,260, filed Sep. 20, 2020.

Cannabichromene Derivative—Cannabichromene-o-acetate

The disclosure generally relates to cannabichromene-o-acetate, methods of making and compositions comprising cannabichromene-o-acetate:

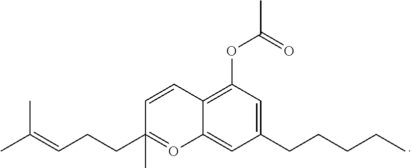

The compound may be of Formula II.

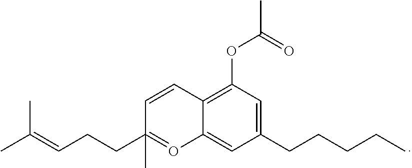

Cannabichromene (CBC) contains a hydroxyl group that can be functionalized to improve permeability across the blood-brain barrier. The inventor surprisingly discovered that functionalizing the hydroxyl group on cannabichromene to form a cannabichromene-o-acetate (Formula I) improved the half-life, potency, and/or the permeability across the blood-brain barrier. Additionally, cannabichromene-o-acetate (Formula II) showed improved the half-life, potency, and/or the permeability across the blood-brain barrier Further, the chemistry of cannabinoids, including cannabichromene, is unpredictable and difficult to modify. Little is known about the physiological effects of cannabichromene and its derivatives.

### Compositions

A composition may comprise cannabichromene-o-acetate. The composition comprising cannabichromene-o-acetate (Formula I) may be a pharmaceutical composition. The composition comprising cannabichromene-o-acetate (Formula I) may be an edible composition. A composition may comprise about 1 nanogram (ng) to about 1 gram of cannabichromene-o- acetate (Formula I).

The effective amount of cannabichromene-o-acetate (Formula I) in a composition may range from about 1 nanogram (ng) to 1 gram (g).

The effective amount of cannabichromene-o-acetate (Formula I) in a composition may be about 1 ng to 1,000 ng. The effective amount may be between about 1 ng and 100 ng, 10 ng and 500 ng, 200 ng and 800 ng, or 250 ng and 750 ng.

The effective amount of cannabichromene-o-acetate (Formula I) in a composition may be about 1 ng, 2 ng, 3 ng, 4 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 21 ng, 22 ng, 23 ng, 24 ng, 25 ng, 26 ng, 27 ng, 28 ng, 29 ng, 30 ng, 31 ng, 32 ng, 33 ng, 34 ng, 35 ng, 36 ng, 37 ng, 38 ng, 39 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 101 ng, 102 ng, 103 ng, 104 ng, 105 ng, 106 ng, 107 ng, 108 ng, 109 ng, 110 ng, 111 ng, 112 ng, 113 ng, 114 ng, 115 ng, 116 ng, 117 ng, 118 ng, 119 ng, 120 ng, 121 ng, 121 ng, 123 ng, 124 ng, 125 ng, 126 ng, 127 ng, 128 ng, 129 ng, 130 ng, 131 ng, 132 ng, 133 ng, 134 ng, 135 ng, 136 ng, 137 ng, 138 ng, 139 ng, 140 ng, 141 ng, 142 ng, 143 ng, 144 ng, 145 ng, 146 ng, 147 ng, 148 ng, 149 ng, 150 ng, 151 ng, 152 ng, 153 ng, 154 ng, 155 ng, 156 ng, 157 ng, 158 ng, 159 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, 400 ng, 410 ng, 420 ng, 430 ng, 440 ng, 450 ng, 460 ng, 470 ng, 480 ng, 490 ng, 500 ng, 525 ng, 550 ng, 575 ng, 600 ng, 625 ng, 650 ng, 675 ng, 700 ng, 725 ng, 750 ng, 775 ng, 800 ng, 825 ng, 850 ng, 875 ng, 900 ng, 950 ng, or 975 ng.

The effective amount of cannabichromene-o-acetate (Formula I) in a composition may between about 1 ng and 100 ng, 10 ng and 250 ng, 100 ng and 500 ng, 250 ng and 500 ng, 300 ng and 900 ng, 400 ng and 600 ng, 125 ng and 725 ng, or 200 ng and 800 ng.

The effective amount of cannabichromene-o-acetate (Formula I) in the composition may be about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 950 mg, or 975 mg.

The effective amount of cannabichromene-o-acetate (Formula I) in a composition may be about 1 mg to 1,000 mg. The effective amount may be between about 1 mg and 100 mg, 10 mg and 500 mg, 200 mg and 800 mg, or 250 mg and 750 mg. The effective amount of cannabichromene-o-acetate (Formula I) in a composition may between about 1 mg and 100 mg, 10 mg and 250 mg, 100 mg and 500 mg, 250 mg and 500 mg, 300 mg and 900 mg, 400 mg and 600 mg, 125 mg and 725 mg, or 200 mg and 800 mg.

The effective amount of cannabichromene-o-acetate (Formula I) in the composition may be about 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g, 112 g, 113 g, 114 g, 115 g, 116 g, 117 g, 118 g, 119 g, 120 g, 125 g, 130 g, 135 g, 140 g, 145 g, 150 g, 155 g, 160 g, 165 g, 170 g, 175 g, 180 g, 185 g, 190 g, 195 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 525 g, 550 g, 575 g, 600 g, 625 g, 650 g, 675 g, 700 g, 725 g, 750 g, 775 g, 800 g, 825 g, 850 g, 875 g, 900 g, 950 g, or 975 g.

The effective amount of cannabichromene-o-acetate (Formula I) in the composition may be about 1 g to 1,000 g. The effective amount may be between about 1 g and 100 g, 10 g and 500 g, 200 g and 800 g, or 250 g and 750 g. The effective amount of cannabichromene-o- acetate (Formula I) in a composition may between about 1 g and 100 g, 10 g and 250 g, 100 g and 500 g, 250 g and 500 g, 300 g and 900 g, 400 g and 600 g, 125 g and 725 g, or 200 g and 800 g.

The cannabichromene-o-acetate (Formula I) compositions described herein may be administered intravesicularly, topically, orally, rectally, vaginally, topically, nasally, or via inhalation. The cannabichromene-o-acetate (Formula I) compositions described herein may be administered orally, optionally in the form of a liquid.

The cannabichromene-o-acetate (Formula I) composition may further comprise a carrier, optionally a physiologically acceptable carrier or excipient to prepare a pharmaceutical composition. The carrier and composition can be sterile. The pharmaceutical preparations can, if desired, be mixed with auxiliary agents, e.g., lubricants, preservatives, stabilizers, wetting agents, emulsifiers, salts for influencing osmotic pressure, buffers that do not deleteriously react with the cannabichromene-o-acetate (Formula I).

The compositions described herein may be formulated as a pharmaceutical composition comprising cannabichromene-o-acetate (Formula I) and a lipid, optionally a fatty acid, oil, or combination thereof. Pharmaceutically acceptable carriers include, but are not limited to, excipient, lubricant, antioxidant, emulsifier, thickening, stabilizer, solvent, diluent, buffer, vehicle, or a combination thereof.

A composition may comprise cannabichromene-o-acetate. The composition comprising cannabichromene-o-acetate (Formula II) may be a pharmaceutical composition. The composition comprising cannabichromene-o-acetate (Formula II) may be an edible composition. A composition may comprise about 1 nanogram (ng) to about 1 gram of cannabichromene-o- acetate (Formula II).

The effective amount of cannabichromene-o-acetate (Formula II) in a composition may range from about 1 nanogram (ng) to 1 gram (g).

The effective amount of cannabichromene-o-acetate (Formula II) in a composition may be about 1 ng to 1,000 ng. The effective amount may be between about 1 ng and 100 ng, 10 ng and 500 ng, 200 ng and 800 ng, or 250 ng and 750 ng.

The effective amount of cannabichromene-o-acetate (Formula II) in a composition may be about 1 ng, 2 ng, 3 ng, 4 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 21 ng, 22 ng, 23 ng, 24 ng, 25 ng, 26 ng, 27 ng, 28 ng, 29 ng, 30 ng, 31 ng, 32 ng, 33 ng, 34 ng, 35 ng, 36 ng, 37 ng, 38 ng, 39 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 101 ng, 102 ng, 103 ng, 104 ng, 105 ng, 106 ng, 107 ng, 108 ng, 109 ng, 110 ng, 111 ng, 112 ng, 113 ng, 114 ng, 115 ng, 116 ng, 117 ng, 118 ng, 119 ng, 120 ng, 121 ng, 121 ng, 123 ng, 124 ng, 125 ng, 126 ng, 127 ng, 128 ng, 129 ng, 130 ng, 131 ng, 132 ng, 133 ng, 134 ng, 135 ng, 136 ng, 137 ng, 138 ng, 139 ng, 140 ng, 141 ng, 142 ng, 143 ng, 144 ng, 145 ng, 146 ng, 147 ng, 148 ng, 149 ng, 150 ng, 151 ng, 152 ng, 153 ng, 154 ng, 155 ng, 156 ng, 157 ng, 158 ng, 159 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, 400 ng, 410 ng, 420 ng, 430 ng, 440 ng, 450 ng, 460 ng, 470 ng, 480 ng, 490 ng, 500 ng, 525 ng, 550 ng, 575 ng, 600 ng, 625 ng, 650 ng, 675 ng, 700 ng, 725 ng, 750 ng, 775 ng, 800 ng, 825 ng, 850 ng, 875 ng, 900 ng, 950 ng, or 975 ng.

The effective amount of cannabichromene-o-acetate (Formula II) in a composition may between about 1 ng and 100 ng, 10 ng and 250 ng, 100 ng and 500 ng, 250 ng and 500 ng, 300 ng and 900 ng, 400 ng and 600 ng, 125 ng and 725 ng, or 200 ng and 800 ng.

The effective amount of cannabichromene-o-acetate (Formula II) in the composition may be about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 950 mg, or 975 mg.

The effective amount of cannabichromene-o-acetate (Formula II) in a composition may be about 1 mg to 1,000 mg. The effective amount may be between about 1 mg and 100 mg, 10 mg and 500 mg, 200 mg and 800 mg, or 250 mg and 750 mg. The effective amount of cannabichromene-o-acetate (Formula I) in a composition may between about 1 mg and 100 mg, 10 mg and 250 mg, 100 mg and 500 mg, 250 mg and 500 mg, 300 mg and 900 mg, 400 mg and 600 mg, 125 mg and 725 mg, or 200 mg and 800 mg.

The effective amount of cannabichromene-o-acetate (Formula II) in the composition may be about 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g, 112 g, 113 g, 114 g, 115 g, 116 g, 117 g, 118 g, 119 g, 120 g, 125 g, 130 g, 135 g, 140 g, 145 g, 150 g, 155 g, 160 g, 165 g, 170 g, 175 g, 180 g, 185 g, 190 g, 195 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 525 g, 550 g, 575 g, 600 g, 625 g, 650 g, 675 g, 700 g, 725 g, 750 g, 775 g, 800 g, 825 g, 850 g, 875 g, 900 g, 950 g, or 975 g.

The effective amount of cannabichromene-o-acetate (Formula II) in the composition may be about 1 g to 1,000 g. The effective amount may be between about 1 g and 100 g, 10 g and 500 g, 200 g and 800 g, or 250 g and 750 g. The effective amount of cannabichromene-o-acetate (Formula I) in a composition may between about 1 g and 100 g, 10 g and 250 g, 100 g and 500 g, 250 g and 500 g, 300 g and 900 g, 400 g and 600 g, 125 g and 725 g, or 200 g and 800 g.

The cannabichromene-o-acetate (Formula II) compositions described herein may be administered intravesicularly, topically, orally, rectally, vaginally, topically, nasally, or via inhalation. The cannabichromene-o-acetate (Formula II) compositions described herein may be administered orally, optionally in the form of a liquid.

The cannabichromene-o-acetate (Formula II) composition may further comprise a carrier, optionally a physiologically acceptable carrier or excipient to prepare a pharmaceutical composition. The carrier and composition can be sterile. The pharmaceutical preparations can, if desired, be mixed with auxiliary agents, e.g., lubricants, preservatives, stabilizers, wetting agents, emulsifiers, salts for influencing osmotic pressure, buffers that do not deleteriously react with the cannabichromene-o-acetate (Formula II).

The compositions described herein may be formulated as a pharmaceutical composition comprising cannabichromene-o-acetate (Formula II) and a lipid, optionally a fatty acid, oil, or combination thereof. Pharmaceutically acceptable carriers include, but are not limited to, excipient, lubricant, antioxidant, emulsifier, thickening, stabilizer, solvent, diluent, buffer, vehicle, or a combination thereof.

A composition may comprise cannabichromene-o-acetate. The composition comprising cannabichromene-o-acetate may be a pharmaceutical composition. The composition comprising cannabichromene-o-acetate may be an edible composition. A composition may comprise about 1 nanogram (ng) to about 1 gram of cannabichromene-o-acetate.

The effective amount of cannabichromene-o-acetate in a composition may range from about 1 nanogram (ng) to 1 gram (g).

The effective amount of cannabichromene-o-acetate in a composition may be about 1 ng to 1,000 ng. The effective amount may be between about 1 ng and 100 ng, 10 ng and 500 ng, 200 ng and 800 ng, or 250 ng and 750 ng.

The effective amount of cannabichromene-o-acetate in a composition may be about 1 ng, 2 ng, 3 ng, 4 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 21 ng, 22 ng, 23 ng, 24 ng, 25 ng, 26 ng, 27 ng, 28 ng, 29 ng, 30 ng, 31 ng, 32 ng, 33 ng, 34 ng, 35 ng, 36 ng, 37 ng, 38 ng, 39 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 101 ng, 102 ng, 103 ng, 104 ng, 105 ng, 106 ng, 107 ng, 108 ng, 109 ng, 110 ng, 111 ng, 112 ng, 113 ng, 114 ng, 115 ng, 116 ng, 117 ng, 118 ng, 119 ng, 120 ng, 121 ng, 121 ng, 123 ng, 124 ng, 125 ng, 126 ng, 127 ng, 128 ng, 129 ng, 130 ng, 131 ng, 132 ng, 133 ng, 134 ng, 135 ng, 136 ng, 137 ng, 138 ng, 139 ng, 140 ng, 141 ng, 142 ng, 143 ng, 144 ng, 145 ng, 146 ng, 147 ng, 148 ng, 149 ng, 150 ng, 151 ng, 152 ng, 153 ng, 154 ng, 155 ng, 156 ng, 157 ng, 158 ng, 159 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng, 200 ng, 210 ng, 220 ng, 230 ng, 240 ng, 250 ng, 260 ng, 270 ng, 280 ng, 290 ng, 300 ng, 310 ng, 320 ng, 330 ng, 340 ng, 350 ng, 360 ng, 370 ng, 380 ng, 390 ng, 400 ng, 410 ng, 420 ng, 430 ng, 440 ng, 450 ng, 460 ng, 470 ng, 480 ng, 490 ng, 500 ng, 525 ng, 550 ng, 575 ng, 600 ng, 625 ng, 650 ng, 675 ng, 700 ng, 725 ng, 750 ng, 775 ng, 800 ng, 825 ng, 850 ng, 875 ng, 900 ng, 950 ng, or 975 ng.

The effective amount of cannabichromene-o-acetate in a composition may between about 1 ng and 100 ng, 10 ng and 250 ng, 100 ng and 500 ng, 250 ng and 500 ng, 300 ng and 900 ng, 400 ng and 600 ng, 125 ng and 725 ng, or 200 ng and 800 ng.

The effective amount of cannabichromene-o-acetate in the composition may be about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 950 mg, or 975 mg.

The effective amount of cannabichromene-o-acetate in a composition may be about 1 mg to 1,000 mg. The effective amount may be between about 1 mg and 100 mg, 10 mg and 500 mg, 200 mg and 800 mg, or 250 mg and 750 mg. The effective amount of cannabichromene-o- acetate in a composition may between about 1 mg and 100 mg, 10 mg and 250 mg, 100 mg and 500 mg, 250 mg and 500 mg, 300 mg and 900 mg, 400 mg and 600 mg, 125 mg and 725 mg, or 200 mg and 800 mg.

The effective amount of cannabichromene-o-acetate in the composition may be about 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g, 112 g, 113 g, 114 g, 115 g, 116 g, 117 g, 118 g, 119 g, 120 g, 125 g, 130 g, 135 g, 140 g, 145 g, 150 g, 155 g, 160 g, 165 g, 170 g, 175 g, 180 g, 185 g, 190 g, 195 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 525 g, 550 g, 575 g, 600 g, 625 g, 650 g, 675 g, 700 g, 725 g, 750 g, 775 g, 800 g, 825 g, 850 g, 875 g, 900 g, 950 g, or 975 g.

The effective amount of cannabichromene-o-acetate in the composition may be about 1 g to 1,000 g. The effective amount may be between about 1 g and 100 g, 10 g and 500 g, 200 g and 800 g, or 250 g and 750 g. The effective amount of cannabichromene-o-acetate in a composition may between about 1 g and 100 g, 10 g and 250 g, 100 g and 500 g, 250 g and 500 g, 300 g and 900 g, 400 g and 600 g, 125 g and 725 g, or 200 g and 800 g.

The cannabichromene-o-acetate compositions described herein may be administered intravesicularly, topically, orally, rectally, vaginally, topically, nasally, or via inhalation. The cannabichromene-o-acetate compositions described herein may be administered orally, optionally in the form of a liquid.

The cannabichromene-o-acetate composition may further comprise a carrier, optionally a physiologically acceptable carrier or excipient to prepare a pharmaceutical composition. The carrier and composition can be sterile. The pharmaceutical preparations can, if desired, be mixed with auxiliary agents, e.g., lubricants, preservatives, stabilizers, wetting agents, emulsifiers, salts for influencing osmotic pressure, buffers that do not deleteriously react with the cannabichromene-o-acetate.

The compositions described herein may be formulated as a pharmaceutical composition comprising cannabichromene-o-acetate and a lipid, optionally a fatty acid, oil, or combination thereof. Pharmaceutically acceptable carriers include, but are not limited to, excipient, lubricant, antioxidant, emulsifier, thickening, stabilizer, solvent, diluent, buffer, vehicle, or a combination thereof.

The compositions described herein may further comprise an antioxidant. Pharmaceutically acceptable antioxidants included, but are not limited to ascorbic acid (vitamin C), glutathione, lipoic acid, uric acid, carotenes, including β-carotene and retinol (vitamin A), α-tocopherol (vitamin E), ubiquinol (coenzyme Q), butylated hydroxyanisole (BHA), butylated hydroxytoluene (BHT), propyl gallate (PG), tert-butylhydroquinone (TBHQ), lutein, selenium, manganese, zeaxanthin, or a combination thereof.

The compositions described herein may further comprise a carrier. Pharmaceutically acceptable carriers are well known in the art and include, for example, vehicles such as glycols, glycerol, oils such as olive oil or organic esters. Pharmaceutically acceptable carriers may be a liquid, including but not limited to oils, including those of petroleum, animal, vegetable or synthetic origin, such as peanut oil, soybean oil, mineral oil, and sesame oil. The pharmaceutical carriers may be gum acacia, gelatin, starch paste, talc, keratin, or colloidal silica. In addition, auxiliary, stabilizing, thickening, lubricating and coloring agents may be used. Further, nutritional ingredients may be included in the pharmaceutical composition. For example, sugars and/or amino acids may be admixed into the pharmaceutical composition. Compositions and methods for compounding cannabinoid derivatives are described in U.S. patent application Ser. No. 17/025,260, filed Sep. 20, 2020.

The compositions described herein may further comprise a fatty acid. In an embodiment, the fatty acid may be arachidic acid, arachidonic acid, behenic acid, bras sidic acid, butyric acid, capric acid, caproic acid, caproleic acid, caprylic acid, cerotic acid, dihomo-γ-linolenic acid (DGLA), docosahexaenoic acid (DHA), docosapentaenoic acid (DPA), eicosapentaenoic acid (EPA), elaidic acid, eurcic acid, gadoleic acid, lauric acid, lauroleic acid, lignoceric acid, linoelaidic acid, linoleic acid, α-linoleic acid, γ-linoleic acid, mead acid, myristic acid, myristoleic acid, nervonic acid, oleic acid, palmitic acid, palmitoleic acid, pinolenic acid (columbinic acid), sapienic acid, stearic acid, vaccenic acid, α-linoleic acid, or a combination thereof.

In an embodiment, composition may comprise an oil. The oil may be a vegetable oil. The vegetable oil may be soybean oil, corn oil, sunflower oil, hemp seed oil, coconut oil, olive oil, canola oil, cottonseed oil, palm oil, peanut oil, safflower oil, sesame oil, or a mixture thereof. The oil may be a nut oil. The nut oil may be almond oil, beech nut oil, cashew oil, hazelnut oil, macadamia oil, mongongo nut oil (manketti oil), pecan oil, pistachio oil, walnut oil, pumpkin seed oil, or a mixture thereof. The hemp seed oil may be extracted using the systems and methods described in U.S. patent application Ser. No. 17/068,092, filed Oct. 12, 2020.

Other examples of suitable pharmaceutical carriers are described in Remington's Pharmaceutical Sciences (Alfonso Gennaro ed., Krieger Publishing Company (1997); Remington's: The Science and Practice of Pharmacy, 21′ Ed. (Lippincot, Williams & Wilkins (2005); Modern Pharmaceutics, vol. 121 (Gilbert Banker and Christopher Rhodes, CRC Press (2002). Where appropriate, the cannabichromene-o-acetate, the compound of Formula I, the compound of Formula II can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. For example, the compositions described herein may be formulated for time-release or delay-release of the cannabichromene-o-acetate (Formula I). In an embodiment, the the cannabichromene-o-acetate (Formula I) may be formulated with an oil as a liquid for oral administration.

The cannabichromene-o-acetate composition may further comprise a cannabinoid. The cannabinoid may be cannabinol, tetrahydrocannabinol (THC), cannabidiol (CBD), cannabidiol acid (CBDA), cannabigerol, cannabinol, cannabichromene, cannabigerivarin, tetrahydrocannabivarin, cannabidivarin, cannabichromevarin, or a mixture thereof. It should be appreciated that the extract may comprise a cannabinoid acid, including but not limited to cannabigerolic acid, cannabidiol acid (CBDA), Δ9-tetrahydrocannabinolic acid (THCA), cannabidiolic acid, cannabichromenenic acid, cannabigerovarinic acid, tetrahydrocanabivarinic acid, cannabidivarinic acid, cannabichromevarinic acid, or a mixture thereof.

The cannabichromene-o-acetate composition may further comprise a terpenes including but not limited to alpha bisabolol, alpha phellandrene, alpha pinene, beta caryophyllene, beta pinene, cadinene, camphene, camphor, citral, citronellol, delta 3 carene, eucalyptol, eugenol, gamma terpinene, geraniol, humulene, limonene, linalool, nerol, nerolidol, ocimene, para-cymene, phytol, pulegone, terpineol, terpinolene, valencene, or mixtures thereof.

The cannabichromene-o-acetate composition may be substantially free of lipids. For example, the cannabinoid extract may comprise less than about 0.001% to 1% lipids by weight.

The cannabichromene-o-acetate composition can comprise less than 0.5%, 0.1%, 0.01%, or 0.001% w/w pesticides, fungicides, fertilizers, and mixtures thereof. For example, the cannabichromene-o-acetate composition can comprise a concentration of pesticides or fungicides ranging from about 0 ppm to 10 ppm.

Additionally, the cannabichromene-o-acetate may be substantially free of cannabis flavonoids including but are not limited to quercetin, luteolin, kaempferol, cannaflavin A, and apigenin. For example, the cannabichromene-o-acetate composition may comprises less than 0.1% to 1% w/w of cannabis flavonoids including but are not limited to quercetin, luteolin, kaempferol, cannaflavin A, and apigenin.

Synthesis of cannabichromene-o-acetate

The cannabichromene-o-acetate may be synthesized from cannabichromene. The cannabichromene may be obtained by the methods known in the art and those methods and systems described in U.S. patent application Ser. No. 16/935,589, filed Jul, 22, 2020. Post-processing methods are described in U.S. Pat. No. 11,148,988. The cannabichromene may be in the form of a crystal, optionally a powdered crystal.

To synthesize cannabichromene-o-acetate from cannabichromene, cannabichromene crystals are placed into a reaction vessel (e.g.,1000 mL Erlenmeyer Flask), optionally with agitation, e.g., with a magnetic stir bar. The reaction vessel, optionally with agitation, is heated to between about 75° C. and 150° C., optionally about 100° C., to melt the cannabichromene into liquid. Liquid cannabichromene presents as an amber liquid. Acetic anhydride are added to the solution at about a 1:1 molar ratio and stirred into solution. Acetic anhydrides, and other suitable anhydrides, may be added in molar ratios between about 1:1 and 1:100. Upon reaching solution homogeneity, a sufficient amount of concentrated sulfuric acid (18M) (e.g., about 5-10 drops) are added into the solution and stirred at about 75° C. and 150° C., optionally about 100° C., for about between 1 and 30 minutes, optionally about 10-15 minutes. Optionally, other strong acids may be used, for example hydrochloric acid, nitric acid, hydrobromic acid, hydroiodic acid, perchloric acid, or chloric acid in a concentration of between about 10M to 20M. e.g., about 18M. The flask is removed from heat and cooled to just above room temperature (20-25 ° C.). 400 mL of hexane and 400 mL of water (i.e., 1:1 ratio by volume) are added to the flask, and the flask is placed on a non-heated stir plate and stir vigorously for 10 minutes. Hexane may also be added in a ratio between about 1:1 and 1:100. The solution is transferred to a 1000 mL separatory funnel to allow for liquid-liquid separation of the organic and aqueous phases.

The respective aqueous and organic phases are drained into separate receptacles and the organic phase (containing the created molecule) is placed into a freezer capable of achieving at least about −5° C., optinally a lower temperature, e.g., −20° C. The final product may begin crystallizing within minutes. Once complete, the solid crystals from the organic solution are filtered over filter paper and washed several times with cold hexane to finish. Once washing is complete, the crystalline solids may be dried, for example, placed in a vacuum oven and evacuated for several hours, e.g., 1-12 hours, at a temperature at about between 30° C. and 60° C., optionally about 45° C.

In another embodiment, the solution is transferred to a 1000 mL separatory funnel to allow for liquid-liquid separation of the organic and aqueous phases. The respective aqueous and organic phases are drained into separate receptacles and the organic phase (containing the created molecule) is placed into a rotary evaporator for recovery of the final product.

Further embodiments of the present invention will now be described with reference to the following examples. The examples contained herein are offered by way of illustration and not by any way of limitation.

### EXAMPLES

The methods described herein will now be described with reference to the following examples. The examples contained herein are offered by way of illustration and not by any way of limitation.

### Example 1

### Synthsis of Cannabichromene-O-Acetate

100 grams (0.322 mol) of cannabichromene (CBC) crystals are placed into a 1000 mL Erlenmeyer Flask with a magnetic stir bar. The flask is placed on a heated stir plate and heated to 100° C., allowing the crystal solids to melt into an amber liquid. 33.2 grams (0.325 mol) of acetic anhydride are added to the solution and stirred into solution. Upon reaching solution homogeneity, 5-10 drops of concentrated sulfuric acid (18M) are added into the solution and stirred at 100° C. for 10-15 minutes. The flask is removed from heat and cooled to just above room temperature (20-25 ° C.). 400 mL of hexane and 400 mL of water (i.e., 1:1 ratio by volume) are added to the flask, and the flask is placed on a non-heated stir plate and stir vigorously for 10 minutes. Other ratios between the organic solvent (hexane) and aqueous solvent (water) may be used, e.g., between about 1:1 and 1: 100. The solution is transferred to a 1000 mL separatory funnel to allow for liquid-liquid separation of the organic and aqueous phases. The respective aqueous and organic phases are drained into separate receptacles and the organic phase (containing the created molecule) is placed into a rotary evaporator for recovery of the final product.

An exemplary synthesis pathway is shown below:

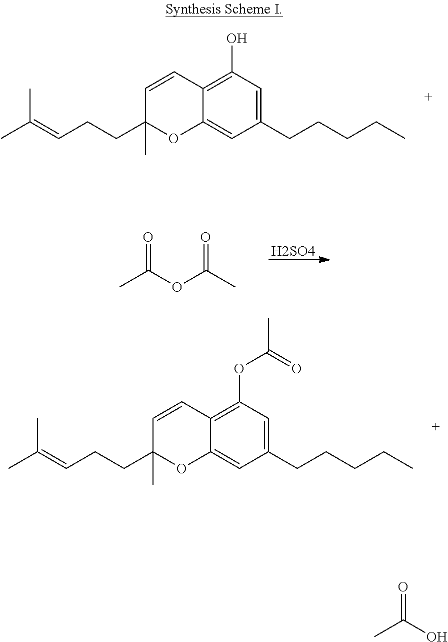

An exemplary synthesis pathway is shown below:

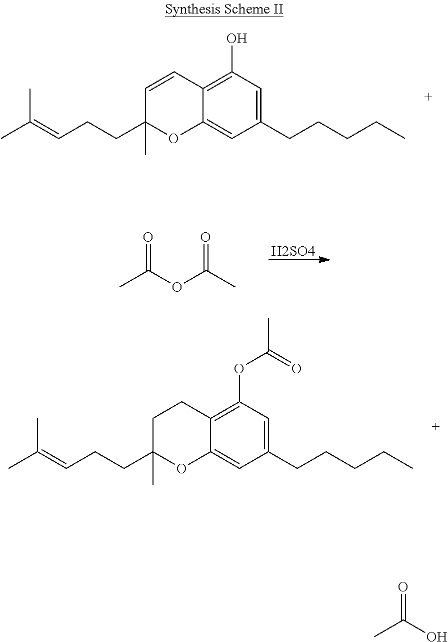

The inventors found that the synthesis schemes I and II provided an unexpected high yield of cannabichromene-o-acetate.

### Example 2

### Treatment of Melanoma

A patient suffering from melanoma may ingest cannabichromene-o-acetate orally. The cannabichromene-o-acetate may work faster and have longer lasting effects than similar dosages of cannabidiol (CBD). The cannabichromene-o-acetate composition may have improved effects as compared to similar dosages of Tetrahydrocannabinol (THC) without the psychoactive side-effects.

Although the subject matter disclosed herein has been described in some detail by way of illustration and example for purposes of clarity of understanding, it should be understood that certain changes and modifications can be practiced within the scope of the appended claims. Modifications of the above-described methods would be understood in view of the foregoing disclosure or made apparent with routine practice or implementation of the described methods to persons of skill in extraction chemistry; extraction processing, mechanical engineering, and/or related fields are intended to be within the scope of the following claims.

All publications (e.g., non-patent literature), patents, patent application publications, and patent applications mentioned in this specification are indicative of the level of skill of those skilled in the art to which this disclosure pertains. All such publications (e.g., non-patent literature), patents, patent application publications, and patent applications are herein incorporated by reference to the same extent as if each individual publication, patent, patent application publication, or patent application was specifically and individually indicated to be incorporated by reference.

While the foregoing methods have been described in connection with this disclosure, it is not to be limited thereby but is to be limited solely by the scope of the claims which follow.

1-86. (canceled)

87. Cannabichromene-o-acetate.

88. The cannabichromene-o-acetate of claim 87, wherein the cannabichromene-o-acetate is of Formula I:

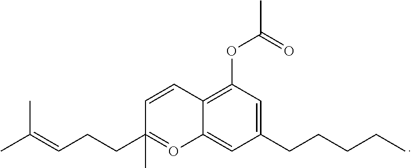

89. A composition comprising the cannabichromene-o-acetate of claim 87.

90. The composition of claim 89, wherein the composition comprises between about 1 ng and 1,000 mg of the compound of Formula I.

91. The composition of claim 89, wherein the composition further comprises an excipient, lubricant, antioxidant, emulsifier, thickening, stabilizer, solvent, diluent, buffer, vehicle, or a combination thereof.

92. The composition of claim 89, wherein the composition further comprises a fatty acid, oil, or combination thereof.

93. The composition of claim 89, wherein the composition further comprises a cannabinoid.

94. The composition of claim 89, wherein the composition further comprises a terpene.

95. The composition of claim 89, wherein the composition is substantially free of lipids.

96. The composition of claim 89, wherein the composition comprises less than 0.3% tetrahydrocannabinol (THC) by weight (w/w).

97. The composition of claim 89, wherein the composition is in the form of a tablet, pill, or capsule, optionally an enteric capsule.

98. The composition of claim 89, wherein the composition is in the form of a liquid, syrup, gel, or gummy.

99. The composition of claim 89, wherein the composition is a pharmaceutical composition.

100. The composition of claim 89, wherein the composition is an edible composition.

101. A method of preparing the compound of claim 87 comprising:
(a) heating cannabichromene to 95-105° C. to form a liquid,
(b) reacting at 95-105° C. the liquid from (a) with acetic anhydride to form a solution,
(c) optionally an acid to the solution from (b),
(d) cooling the solution from (b) or (c) to 20-25° C. to form a cooled solution,
(e) washing the cooled solution from (d) with water and hexane, forming organic and aqueous phases,
(f) separating the organic and aqueous phases, wherein the organic phase comprises the cannabichromene-o-acetate; and
(g) recovering the Cannabichromene-o-acetate from the organic phase.

102. The method of claim 101, wherein the acid is sulfuric acid, hydrochloric acid, nitric acid, hydrobromic acid, hydroiodic acid, perchloric acid, or chloric acid.

103. The method of claim 101, wherein the acid is sulfuric acid.

104. The method of claim 101, wherein one or more of (a), (b), (c), and (e) further comprises stirring.

105. The method of claim 101, wherein in (e), water and hexane are in a 1:1 ratio by volume.

106. The method of claim 101, wherein in (a), the cannabichromene and acetic anhydride are in a molar ratio between about 1:1 and 1:100, optionally a 1:1 molar ratio.
